(19) United States
(12) Patent Application Publication
(10) Pub. No.: US 2013/0197317 A1
(43) Pub. Date: Aug. 1, 2013

(54) Detachably-mountable, compact, light for surgical and diagnostic devices

(71) Applicants: Steven A. Daniel, Fremont, CA (US); Claudio I. Zanelli, Menlo Park, CA (US)

(72) Inventors: Steven A. Daniel, Fremont, CA (US); Claudio I. Zanelli, Menlo Park, CA (US)

(73) Assignee: InLight Medical, Inc.

(21) Appl. No.: 13/385,109

(22) Filed: Feb. 1, 2012

(51) Int. Cl. A61B 1/06

(52) U.S. Cl. 600/249

(57) ABSTRACT

A detachable light for use in combination with surgical and diagnostic devices. The detachable light incorporates multiple functions into a minimum number of elements in the housing of the detachable light. The housing includes aligning gripping elements which align the light to a functional centerline of the surgical or diagnostic device. The housing includes multiple light sources on its exterior surface, where the direction of light output may be adjusted. Exterior surfaces of the detachable light which are in contact with patient tissue are formed from an atraumatic material.

### FIELD

Embodiments of the present invention relate to a detachable, compact, optionally self-powered, light which may be attached to various surgical, diagnostic, and other medical devices, to bring the appropriate lighting upon a tissue which is located internal or external to a patient, where other available lighting is incapable of illuminating the tissue. Lights which make use of the features of the invention may also find application in industrial and consumer environments.

### BACKGROUND

This section describes background subject matter related to the disclosed embodiments of the present invention. There is no intention, either express or implied, that the background art discussed in this section legally constitutes prior art.

Tissues which are internal to the body may be viewed using a number of different imaging techniques which are known in the art. Many of these techniques are too bulky and cumbersome to be helpful during surgery or diagnostic investigations, and are expensive to operate and maintain. Further, in some instances external lighting may fail to provide sufficient illumination or may not be available. There is a need for a light source which properly illuminates a tissue site, both in instances where an internal tissue site cannot be illuminated sufficiently by an external light source, and in instances where an external tissue site cannot be illuminated sufficiently by available external lighting.

The detachable light source needs to provide a luminescent intensity which allows a clinician, for example, to accurately and confidently identify and treat the various normal and abnormal tissues found in the body. The light source needs to provide light within a frequency/color range that permits proper identification of tissue type. The light source needs to be compact and to be easily attachable and detachable from diagnostic instrumentation used by the clinician, or from surgical tools used by a surgeon, for example. A light-generating apparatus which is compact and disposable would be advantageous in view of sterility considerations.

In addition, the power supply to the light-generating apparatus is a major consideration. In some instances a power cord may be run from a surgical power supply line present in a sterile surgical environment. In other instances, where a power cord would hamper the ability of a surgeon or clinician to work, a battery power source which could fit into a pocket of the surgeon or clinician, or a battery source present within the housing of the detachable light is advantageous.

A few illustrative examples of attempts to provide hand-held surgical lights include the following. U.S. Pat. No. 5,283,722 to Koenen et al., issued Feb. 1, 1994, describes a surgical-type glove and illuminator assembly particularly adapted for use by health care professionals when examining or operating upon an anatomical part of a patient. A spotlighting illuminator is securely mounted on the fingers portion of the glove and oriented to project a light beam distally of the glove toward the work surface when the glove is in use. The illuminator may have a self-contained light source, or utilize fiber optic-transmitted light from a light source remote from the glove. (Abstract) U.S. Pat. No. 6,428,180 to Karram et al., issued Aug. 6, 2002 describes a compact, self-powered, selectively-mountable lighting unit which is said to provide light directable by a user to an operation site in a confined space to enable the user to operate a tool therein. (Abstract) However, applicants contend that in 2000, when the utility application was filed for the '180 patent, it was not possible to produce a configuration that was “compact” enough that could also produce enough light for a sufficient time to be able to accomplish a surgery. U.S. Pat. No. 6,540,390 to Toth et al., issued Apr. 1, 2003, describes a hand-held surgical light assembly which provides a light source and a hand piece which is adapted to be grasped and manipulated by a user. (First part of the Abstract) U.S. Pat. No. 7,270,439 to Horrell et al., issued Sep. 18, 2007 describes a compact, self-contained lighting system which is attachable to a surgical tool to enable a user to selectively direct light at a site where the tool is to be applied. The system is said to have a power unit that may contain rechargeable power cells, a malleable electrical connection element, and a light-emitting element powered thereby to emit high intensity white light, preferably from an LED. (First part of the Abstract)

As can be seen from the above descriptions (which can be reviewed in more detail by obtaining a copy of the cited reference) there is a well recognized need for a compact light which is part of a surgical tool, or which may be attached to a surgical tool or diagnostic tool, to provide light at a tissue site which is internal to a patient. Each of the patents referenced above describes a different combination of elements which is used to provide such a light. There are other examples not mentioned because they appear to applicants to be more remote from applicants' invention, or they are cumulative. There may be other examples which are not known by applicants.

### SUMMARY

The primary purpose of a detachable light for use in combination with a surgical or diagnostic device is to provide sufficient light to allow the clinician to accurately and confidently identify or treat various tissue which is located internal or external to a patient, where external lighting is incapable of adequately illuminating the tissue. In some instances where overhead, external lighting is not available, the detachable light may be the only source of light used in a procedure.

In most instances, where a surgical or diagnostic device is used in a room which provides an independent source of overhead light, the detachable light will provide an improvement in lighting which allows the care giver to view the surgical or diagnostic field and make improved medical decisions based on what the care giver sees.

An inadequate luminescent intensity would either cause the care giver to make improper medical decisions or to provide inadequate care. In order to fulfill the requirement for adequate luminescent intensity, a typical embodiment of the detachably-mountable light needs to be capable of producing an illumination in excess of 20,000 Lux or 20,000 lumen per square meter of light at the distal end of the surgical or diagnostic tool. An upper range of luminescent intensity is expected to be about 150,000 Lux, so that the light source is not so bright that the vision of the care giver is overwhelmed, with the result that the ability of the human eye to see is decreased. In particular, the care giver often may need to observe various instrumentation and monitors in addition to the surgical field. Once adjusted to high intensity light, the vision of the care giver may be significantly reduced at normal ambient light levels. As a unit of measure, the Lux is preferred, because this unit may be weighted so the various frequencies of light are measured as perceived by the human eye.

The frequency of the light which is used will depend on the end use application for the detachable light. However, a commonly used Operating Room (OR) white light will be required for many applications. Typically we think of light as being “white light” in general. In fact, there are an infinite number of shades of “white light”' which appear more yellow (warmer color) to more blue (cooler color). The most common embodiments of the invention are capable of providing light within a frequency/color range which is customarily given off by overhead operating room lights. In this way, the user does not have to try to compensate for a difference between the light provided by the OR overhead lights they are accustomed to, or from previous training related to the identification and differentiation of different tissues. In the descriptions herein, the frequency of light is expressed as a function of the light source temperature, and is indicated in degrees Kelvin (° K), a standard unit of measurement in this field. The OR white light typically provides light within a range of between about 3,500° K and about 5,500° K.

Managed heat dissipation is important with respect to the detachable light. Typically high intensity light creates a large amount of heat that must be managed and dissipated. Even LED bulbs, which are typically more efficient than other types of light sources when used to create high intensity light, generate a significant amount of heat which must be dissipated. In most embodiments of the invention, this has been resolved without the need for additional heat sinks (which require space), without the need for highly thermally conductive materials (which are particularly expensive), and without the need for auxiliary cooling (which also requires space).

To manage the heat dissipation, the number of light sources is designed so that each light source operates in a high efficiency operating zone. By not driving the individual light sources into their less efficient operating range, less heat is generated. In addition, the shape of the housing for the light sources (bulbs), can be used to reduce heat generation. It is generally known that circular shapes provide the largest amount of surface area to volume ratio. By controlling the diameter of the housing for the light sources (within the maximum sizing permissible for the detachable light tool or diagnostic tool), improved dissipation for unwanted heat can be achieved while still producing a high intensity light. The use of multiple elements allows additional light to be provided while the unwanted heat is more evenly dissipated around the device housing.

Many of the internal tissues and structures within the body are sensitive and can be damaged by direct contact with the surgical tool or diagnostic tool. The detachable light needs to provide an exterior surface which is not likely to harm tissue upon contact. The exterior surface needs to be a soft atraumatic surface. At the same time, the structure of the housing of the detachable light must provide dimensional stability for the lighting device, including structural and lighting elements, and power sources (whether mounted within the lighting housing or connected to a power cable). In addition, the detachable lighting physical structure must provide for generation of a sufficient spring force for clamping or attaching the lighting housing onto the surgical instrument or diagnostic device.

Thus, the housing of the detachable light advantageously includes a rigid internal structure in combination with a soft atraumatic material on exterior surfaces of the detachable light. The interior of the lighting housing may comprise a ridged (finger-like) internal structure to grip the surgical or diagnostic tool surface and simultaneously provide rigidity of the overall housing structure. Internal ridged members may be used to provide structural integrity, alignment features for the lighting housing relative to the surgical or diagnostic device, and a spring force required to create a clamping function which holds the detachable light in place.

Examples of materials which may be used to form the internal structural features of the light housing include nitinol, stainless steels, beryllium copper, and plastics such as polyetherimide (PEI), polyoxymethylene, polysulfones, poly vinylidene fluoride, nylons, ABS, LCP, and polycarbonate, by way of example and not by way of limitation. Examples of materials which may be used to provide an atraumatic exterior surface on the housing include silicone; urethanes; thermoplastic elastomers, including block copolymers of styrene with butadiene and/or isoprene, and blends of these materials with other thermoplastic materials; and latex, by way of example and not by way of limitation.

A directional alignment of the light or light elements is required to ensure a desired level of luminescence in the target field of view. Embodiments of the invention achieve this by creating a foundation element within the body of the detachable lighting device. The foundation element, which may be based on a centerline, for example and not by way of limitation, allows the lighting elements and the surgical or diagnostic device to share a common geometric reference point (or points). This arrangement helps establish and maintain correct orientation and alignment for each element of the detachable light relative to the surgical or diagnostic tool. This ensures the proper direction (X, Y, Z, and angle) for the light output.

To achieve a fully illuminated field, there are a number of elements which must be satisfied. To have a fully illuminated field of view that is properly sized for optimum viewing, the light sources must have the right combination of source location referenced to the field of view, beam divergence, a common output direction for the source light, and correct beam overlap.

As described above, the location and beam direction can be achieved with the aid of the correct foundation element or elements. Beam divergence can be specified and implemented with lenses used on light source(s). The remaining element, overlapping pattern of the beams, must be achieved in a manner which provides sufficient intensity for the resulting spot size needed for the particular medical application.

With respect to spot size, in one embodiment of the present invention eight discrete points of divergent light output are used. These points of light output can be created in a number of ways, including individual light elements such as LED or a single light source used in conjunction with light pipes. For illustration purposes subsequently herein, and not by way of limitation, a description of a detachable light comprising eight LEDs is provided.

A fully illuminated field is very important. This requires a minimization of shadowing. Typically, shadowing is caused by the structural configuration of the instrument or tool to which the detachable light is attached. A shadow will always be cast when a light source encounters an obstruction. This effect obviously has a negative impact on a care giver's ability to discern details within the surgical field of view. In order to minimize this effect, the points of light output are arranged in such a way as to create a source mirror on the elements of the surgical or diagnostic instrument causing an obstruction, for example. Shadows created by a surgical instrument or diagnostic tool cannot be eliminated by light placement. However, the embodiments of the invention are designed to make use of two or more points of light, to balance the light and dark regions of this effect, allowing the care giver to have a uniform and highly illuminated field of view.

Adjustable beam direction may be obtained by using a balance between semi-ridge but movable mounting of each light element and the correct degree of pliability and dimensional material memory for the distal portion of the housing. “Semi ridge” refers to a condition when there is a device which is able to maintain its shape (for example) and resist minor amounts of applied force without deformation. However, when sufficient force is applied, movement may be achieved. In the present invention, the device may be designed so that the light sources are able to maintain their dimensional stability under minor loading. However, when sufficient force is applied by the operator at particular locations relative to the light sources, the light sources may be moved/adjusted by the user. Once the force is removed, the light sources retain their “new” location or direction on the device. In one embodiment of the invention, this is accomplished by using bendable or malleable light source leads which are connected to a more rigid core which shares or provides the referenced orientation points for the light sources. The more rigid core may be a “C” ring in some instances, for example. There are other devices that provide either a long bendable member such as a “goose neck” or which define joints for adjustment. However these do not allow adjustment of individual light source elements within a multi-light configuration.

While it would be possible for the detachable light to be re-usable after a sterilization process, to minimize the potential for contamination between patients, a single use device is preferable.

### BRIEF DESCRIPTION OF THE DRAWINGS

So that the manner in which the exemplary embodiments of the present invention are attained is clear and can be understood in detail, with reference to the particular description provided above, and with reference to the detailed description of exemplary embodiments, applicants have provided illustrating drawings. It is to be appreciated that drawings are provided only when necessary to understand exemplary embodiments of the invention and that certain well known processes and apparatus are not illustrated herein in order not to obscure the inventive nature of the subject matter of the disclosure.

FIG. 1A shows a first embodiment of a compact detachable light 100. The detachable light includes 8 light sources 104 in the form of LED bulbs, the distribution of which is designed to produce uniform light distribution, thereby reducing shadows in the light cast by the LED bulbs. The housing 102 of the detachable light is formed from a compound which provides a smooth, soft exterior surface, and an interior surface which is sufficiently tacky to stay in place on the exterior of a surgical or diagnostic device (not shown).

FIG. 1B shows a close up of the compact detachable light 100 shown in FIG. 1A, and illustrates a power attachment cord 106 extending through a connection inlet 105 into the housing 102. Power cord 106 may be attached to a power outlet (not shown) such as a wall outlet in a surgical room, for example.

FIG. 1C shows the detachable light 100 shown in FIG. 1A and illustrates a slot 108 through which a surgical or diagnostic device (not shown) may be inserted into the detachable light 100 housing 102.

FIG. 1D shows the detachable light 100 shown in FIG. 1A, and illustrates the expansion 110 of the housing 102, which permits the insertion of varying sizes of diagnostic or surgical devices (not shown).

FIG. 2A shows another embodiment of the compact detachable light 200 which is an alternative shape and structure. The housing 202 is of a triangular shape on the exterior and includes 3 light sources 204. The three light sources 204 surround an opening 203 through which a surgical or diagnostic device (not shown) may extend.

FIG. 2B shows one embodiment of a gripping device 212 which may be present within the interior of housing 202. The gripping device 212 includes a pivotal device 208, such as a sphere with an opening 209 passing through the sphere. The internal surfaces 214 of the opening 209 act as angled faces which can be adjusted at surfaces 215 against the housing 202. Typically a packing element 216 is applied against an exterior surface 217 to hold gripping device 212; inside the interior of housing 202. The packing 216 and pivotal structure 217 are shown as held in position by a retention bar 210. An alternative to use of a gripping device of the kind shown in FIG. 2B would be the use of semi ridge materials interior to the housing 202, where pressure on the exterior of housing 202 may be used to adjust the relative position of light sources 204 relative to a centerline (not shown) through the gripping device.

FIG. 2C shows a side view of the detachable light 200, and illustrates a connection inlet 205.

FIG. 2D illustrates a front view of the detachable light 200, and shows an illuminated area 216 provided by light sources 204 surrounding the opening 203, so that a surface (not shown) toward which a surgical or diagnostic device (not shown) extends will be fully illuminated, as illustrated by area 216.

FIG. 3A shows an embodiment of a compact detachable light 300 which is attached to an electrocautery hand piece 306, for example and not by way of limitation. The detachable light housing 302 includes light sources 304 and an opening 305 through which the tool 306 may extend. The tool 306 includes “on”-“off” switches 308 which are typically used to control the operation of the functional device (such as the electrocautery hand piece shown). The functional device may be battery powered or may be connected to a power source (the power source for the device is not shown in FIG. 3A).

FIG. 3B shows the detachable light 300 of FIG. 3A, and illustrates the presence of batteries 310, located within housing 302, which batteries are used to power the detachable light 300. The detachable light 300 may include an inductive pickup (not shown) that can “sense” energy flow in the tool and activate the battery power to detachable light 300. Addition of the inductive pickup may also be used help to draw power from an active tool such as an electrocautery hand piece to help power the light sources; or to power the light sources entirely (not shown).

FIG. 4A shows an additional embodiment of a compact detachable light 400 which is attached to an electrocautery hand piece 406. The surgical or diagnostic tool 406, includes “on”-“off” switches 408. The housing 402 of detachable light 400 includes three light sources 404, and batteries 410 which power the light sources. As with the embodiment shown in FIG. 3C, the detachable light 400 includes an inductive pickup (not shown) that can “sense” energy flow in the tool and activate the battery power to detachable light 400.

FIG. 4B shows the presence of the batteries 410, located within section 405 of the housing 402.

FIGS. 5A through 5D show a series of views of a detachable light 500 which can fit upon the surface of a large number of different kinds of surgical or diagnostic devices, due to the clamping mechanisms 505 present on the internal surface 503 of the housing 502, in combination with the tapered sidewalls 503 of the detachable light housing 502. The clamping mechanisms 505 are employed to fasten the detachable light 500 to a surgical or diagnostic tool (not shown).

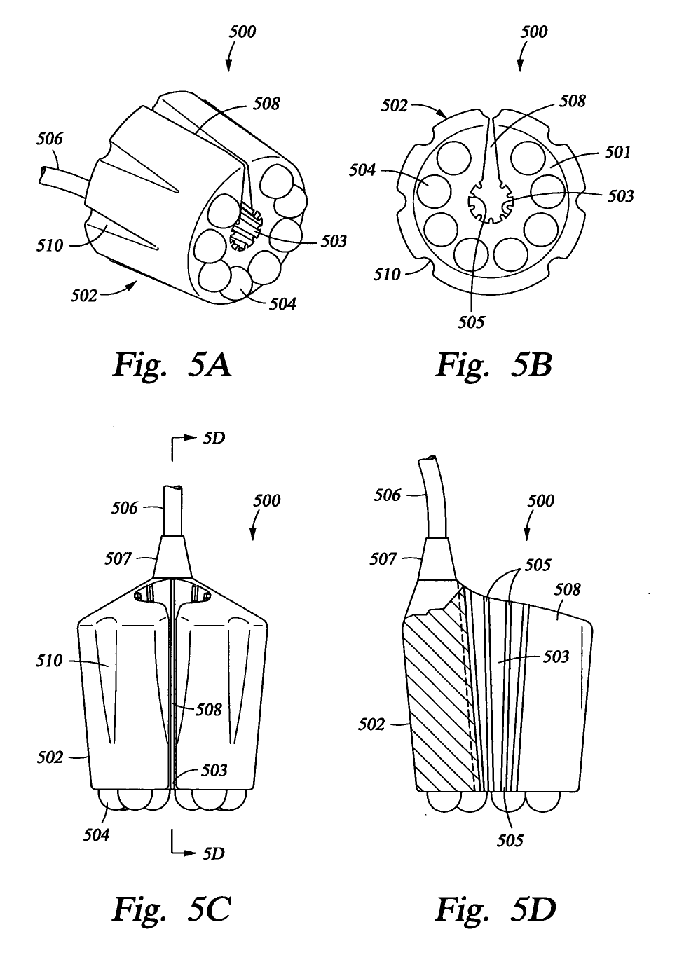

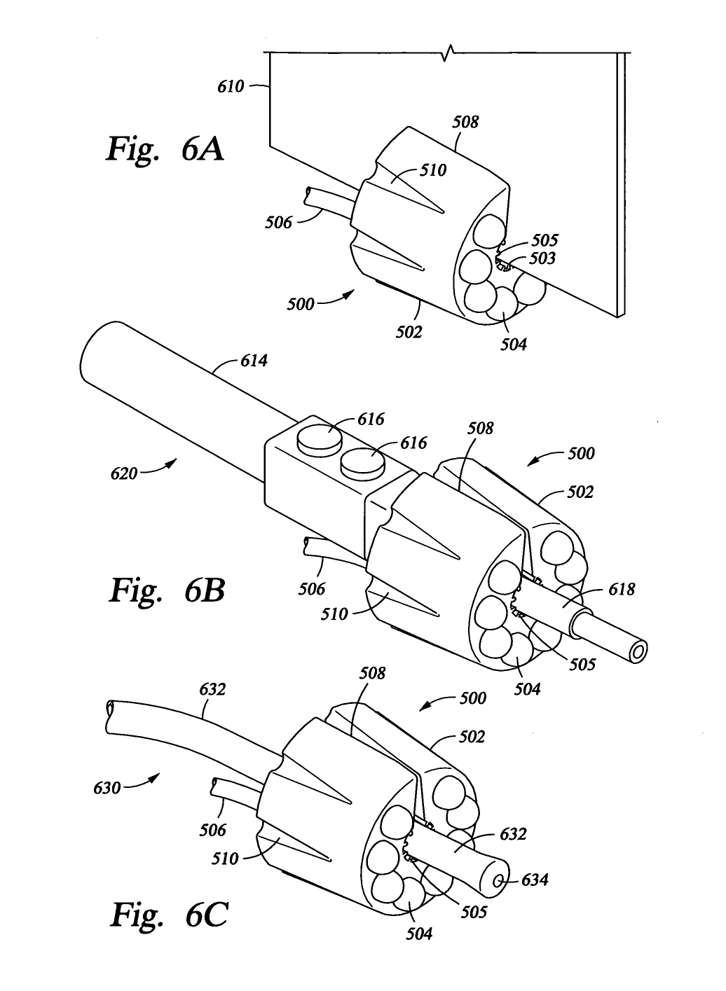

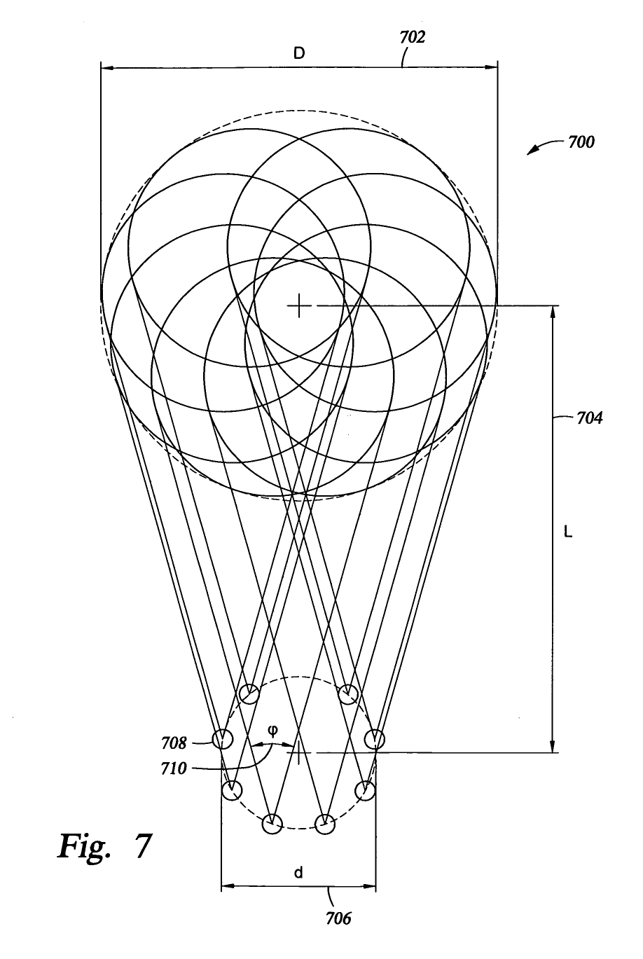

In FIG. 5A the overall design and shape of the detachable light housing 500 is illustrated. The detachable light housing 500 includes a slot 508 which passes all the way through housing 500, and permits the insertion of a surgical or diagnostic tool (not shown) into the interior 503 of housing 500. The exterior surface 502 of housing 500 includes grooves 510 designed to permit the operator of the surgical or diagnostic tool to better grip the housing 500 and adjust the housing 500 on the tool as desired. A power supply cord 506 is shown on the proximal, back end of the detachable light housing 500.

FIG. 5B shows a front view of the detachable light housing 500 illustrated in FIG. 5A, where the tapered grooves 510 are present on the exterior surface 502 of the housing. The slot 508 is present from the exterior surface 502 to the interior surface 503 of the housing 500 to permit insertion of the tool (not shown). The interior surface 503 comprises ridges or “fingers” 505 which are used to conform to and apply pressure to the exterior surface of the tool (not shown). These “fingers” are typically formed of a flexible material which can be compressed to accommodate different sizes and shapes of tools. The light sources 504 are arranged around the outer edge of the proximal face 501 of housing 500 in a manner which reduces shadowing due to the presence of elements of a tool (not shown).

FIG. 5C shows a side view of the detachable light housing 500 which is illustrated in FIG. 5A. The slot 508 which is present for the length of the housing 500 and which passes all the way through from the exterior surface of the housing to the interior surface is illustrated, including the clamping/holding fingers 505.

FIG. 5D shows a “break-away” side view of the detachable light housing 500, clearly showing a conical shape along the interior surface 503 of the housing 500, with a larger opening 503A being present at the proximal, backside 502 of the housing 500 and a smaller opening 503B being present at the distal, front side (not shown) of the housing 500. The power connection inlet 507 is shown at the proximal, backside of the housing 500. Fingers 505 which provide the clamping action to hold a tool (not shown) are also illustrated.

FIGS. 6A through 6C illustrate the lighting housing 500 from FIG. 5 attached to a variety of surgical or diagnostic device surfaces.

FIG. 6A shows the housing 500 attached to a planar surface 610 which may extend from a surgical or diagnostic device (entire device not shown). The internal fingers 505 of the housing hold the planar surface 610 which is inserted through slot 508.

FIG. 6B shows a view of the housing 500 attached over a tool 620 having a handle 614, “on”-“off” switches 616, and a front extension 618. The tool 610 is powered by a power cord 506 which may be attached to a wall outlet (not shown).

FIG. 6C shows the housing 500 attached to an aspirator 630 of the kind used for removing unwanted fluid or smoke from a surgical field. An exterior surface 632 of the aspirator 630 is grasped by fingers 505 on the interior surface of the housing 500. The light sources 504 are designed to have a centerline (not shown) which passes through opening 634 through the interior of the aspirator 630.

FIG. 7 illustrates the considerations which are used in determining the light source spacings which should be used to reduce shadowing for a particular surgical or diagnostic device/tool. The divergence angle is represented by “φ”. “d” represents the diameter of a circle around which the lighting sources are placed. “D” represents a resulting illuminated diameter or spot size at a distance “L” from the light sources. If the divergence angle φ is too great in relation to the distance L, the resulting spot size will be too large, causing a reduction in the illumination intensity or density.

### DETAILED DESCRIPTION OF EXEMPLARY EMBODIMENTS

As a preface to the detailed description, it should be noted that, as used in this specification and the appended claims, the singular forms “a”, “an”, and “the” include plural referents, unless the context clearly dictates otherwise.

When the word “about” is used herein, this is intended to mean that the nominal value presented is precise within ±10%.

The various exemplary embodiment examples of the detachably-mountable surgical (or diagnostic) tool light have a number of basic features in common. In addition, there are some “add-on” features which are needed depending on the particular circumstances of use for a given embodiment.

As previously discussed, the primary purpose of the device is to provide sufficient, evenly distributed, light to allow the user to accurately and confidently visualize an area of otherwise reduced or low light level. In a preferred particular embodiment, a clinician may be enabled to accurately and confidently identify and treat the various normal and abnormal tissues found in the body. Correct lighting allows the care giver to view the surgical field and make medical decisions based on what the care giver sees.

An inadequate luminescent intensity could either cause a care giver to make improper medical decisions or to provide inadequate care. In order to fulfill the requirement for adequate luminescent intensity, a typical embodiment of the detachably-mountable surgical light or diagnostic tool is capable of producing an illumination in excess of 20,000 Lux or 20,000 lumen per square meter of light at the distal end of the surgical light or diagnostic tool. As a unit of measure, the Lux is preferred, because this unit can be used to weight the various frequencies of light as perceived by the human eye. Typically the light intensity ranges between about 20,000 Lux and 150,000 Lux. In many instances, light intensity ranging between about 50,000 Lux and 100,000 Lux provides good results in terms of tissue recognition.

In some situations, a higher or lower level of light is required to provide the best visualization. A rheostat, or another kind of variable power control, which provides an ability to adjust the amount of power supplied to the detachable light, and thus the intensity of the light over a large range, may be made available between the power supply and the detachable light. The adjustment device for the rheostat may be present in the form of a controller which is connected to the light, where the controller is located on a surface which is convenient to the practitioner. In the alternative, the adjustment device may be present as a control knob on the detachable light itself. The practitioner or care giver may adjust the amount of power to the light during a procedure to help provide lighting conditions which improve the recognition of tissue.

The “color” of the light, i.e., the frequency or frequency distribution of the light which is used, will depend on the end use application for the detachable light. However, a commonly used Operating Room (OR) white light is advantageous for many applications. Typically we think of light as being “white light”. In fact, there are an infinite number of shades of ‘white light’ which appear more yellow (warmer color) to more blue (cooler color). The most common embodiments of the invention are capable of providing light within a frequency/color range which is customarily given off by overhead operating room lights. In this way, the user does not have to make undue adjustments or compensate for differences between the light generated by the detachable light and the overhead light the practitioner is accustomed to for purposes of the identification and differentiation of different tissues. In the descriptions herein, the frequency of light is expressed as a function of the light source temperature, and is indicated in degrees Kelvin (° K), a standard unit of measurement in this field. The OR white light typically provides light within a range of between about 3,500° K and about 5,500° K.

The use of light frequencies other than the OR white light may be used for the detection of various conditions such as bile leaks, cancerous tissue, and other medical conditions. Some embodiments of the detachable light may contain more than one frequency of light source bulbs which are used in combination for particular applications. An example of this is the replacement of one or more of the “white” light sources with one or more ultra violet light sources. In yet another embodiment, a filter cap is placed over the emitting face of the light sources to alter color of the emitted light energy as desired.

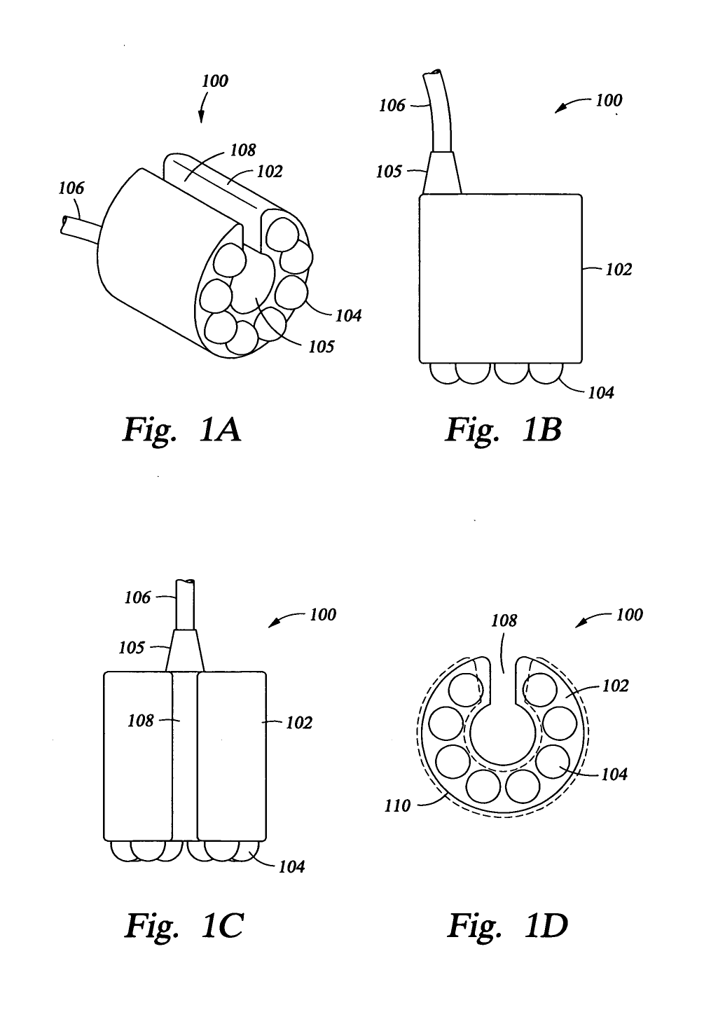

FIG. 1A shows a first embodiment of a compact detachable light 100. The detachable light includes 8 light sources 104 in the form of LED bulbs, the distribution of which is designed to produce uniform light distribution, thereby reducing shadows in the light cast by the LED bulbs. The housing 102 of the detachable light is formed from a compound which provides a smooth, soft exterior surface, and an interior surface which is sufficiently tacky to stay in place on the exterior of a surgical or diagnostic device (not shown). The housing of the detachable light may be formed from a compound which provides a smooth, soft exterior surface, such as that which may be provided by a silicone compound. The interior surface which is sufficiently tacky to stay in place on the exterior of surgical or diagnostic device may be formed from a material such as a polyurethane, for example and not by way of limitation. FIG. 1B shows a close up of the compact detachable light 100 shown in FIG. 1A, and illustrates a power attachment cord 106 extending through a connection inlet 105 into the housing 102. Power cord 106 may be attached to a power outlet (not shown) such as a wall outlet in a surgical room, for example.

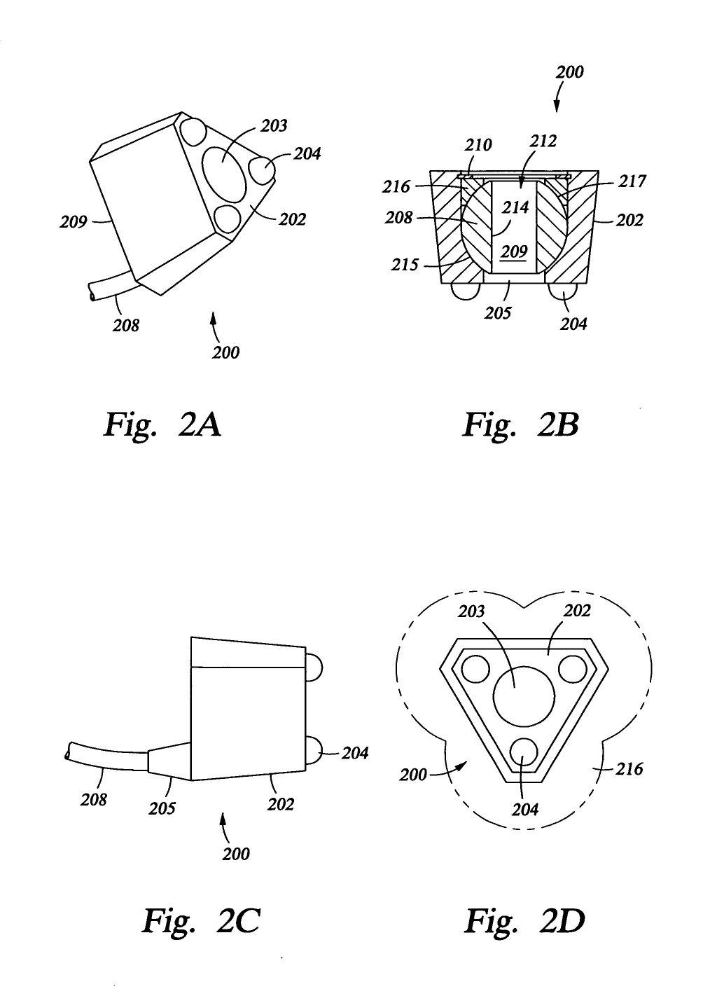

FIG. 2A shows another embodiment of the compact detachable light 200 which is an alternative shape and structure. The housing 202 is of a triangular shape on the exterior and includes 3 light sources 204. The three light sources 204 surround an opening 203 through which a surgical or diagnostic device (not shown) may extend. FIG. 2B shows one embodiment of a gripping device 212 which may be present within the interior of housing 202. The gripping device 212 includes a pivotal device 208, such as a sphere with an opening 209 passing through the sphere. The internal surfaces 214 of the opening 209 act as angled faces which can be adjusted at surfaces 215 against the housing 202. Typically a packing element 216 is applied against an exterior surface 217 to hold gripping device 212; inside the interior of housing 202. The packing 216 and pivotal structure 217 are shown as held in position by a retention bar 210.

FIG. 2C shows a side view of the detachable light 200, and illustrates a connection inlet 205. FIG. 2D illustrates a front view of the detachable light 200, and shows an illuminated area 216 provided by light sources 204 surrounding the opening 203, so that a surface (not shown) toward which a surgical or diagnostic device (not shown) extends will be fully illuminated, in the manner illustrated by area 216.

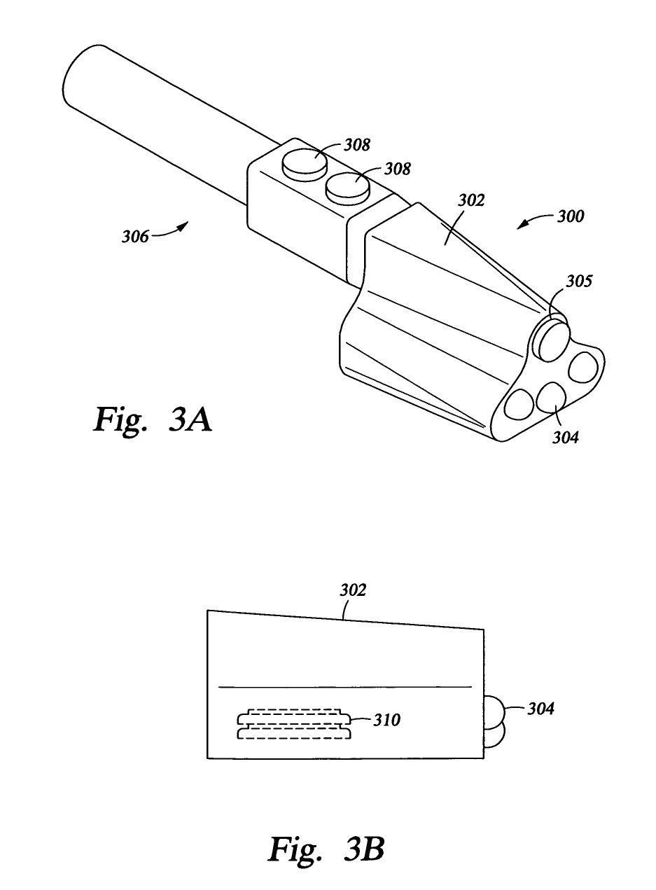

FIG. 3A shows an embodiment of a compact detachable light 300 which is attached to an electrocautery hand piece 306. The detachable light housing 302 includes light sources 304 and an opening 305 through which the tool 306 may extend. The tool 306 includes “on”-“off” switches 308 which are used to turn the tool on or off.

FIG. 3B shows the detachable light 300 of FIG. 3A, and illustrates the presence of batteries 310, located within housing 302, which batteries are used to power the detachable light 300. As previously discussed, there may be a sensing device (not shown) present in the detachable light housing which includes an inductive pick up that can sense energy flow in the tool and activate the battery power to the light sources. In an alternative embodiment, there may be a surface on the exterior of the tool which puts pressure on a pressure sensor inside the detachable light housing to cause the battery power supply to activate.

In yet another embodiment, when the tool to which the detachable light housing is to be attached has a power cord which sends power to the tool, there may be a form of jacketed electrical contacts on an exterior surface of a tool which can be uncovered to provide an electrical supply to electrical contacts on a surface of the detachable light, so that it is not necessary to have a battery to supply power to the detachable light.

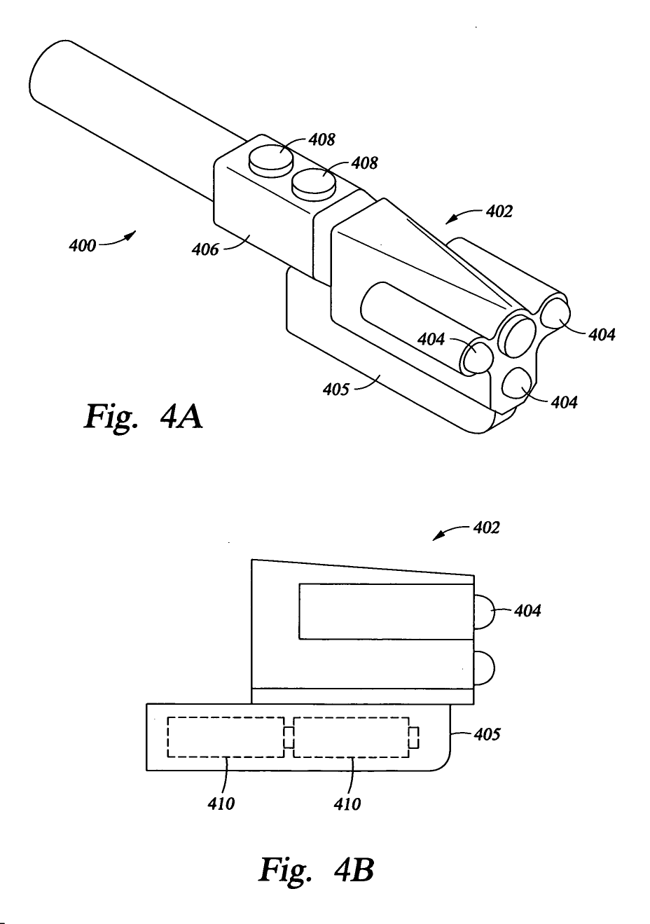

FIG. 4A shows an additional embodiment of a compact detachable light 400 which is attached to an electrocautery hand piece 406. The surgical or diagnostic tool 406, includes “on”-“off” switches 408. The housing 402 of detachable light 400 includes three light sources 404, and batteries 410 which power the light sources. As with the embodiment shown in FIG. 3C, the detachable light 400 may includes an inductive pickup (not shown) that can “sense” energy flow in the tool, or a power pressure button which can activate the battery power to detachable light 400.

FIGS. 5A through 5D show a series of views of a detachable light 500 which has can fit upon the surface of a large number of different kinds of surgical or diagnostic devices, due to the clamping mechanisms 505 present on the internal surface 503 of the housing 502. The clamping mechanisms 505 are employed to fasten the detachable light 500 to a surgical or diagnostic tool (not shown). While the tool may be an electrocautery hand piece or an aspirator, as previously mentioned, these are only examples of tools to which the detachable light may be attached. Other examples include retractors, clamps, and the like, by way of example and not by way of limitation.

In FIG. 5A the overall design and shape of the detachable light housing 500 is illustrated. The detachable light housing 500 includes a slot 508 which passes all the way through housing 500, and permits the insertion of a surgical or diagnostic tool (not shown) into the interior 503 of housing 500. The exterior surface 502 of housing 500 includes grooves 510 designed to permit the operator of the surgical or diagnostic tool to better grip the housing 500 and adjust the housing 500 on the tool as desired. A power supply cord 506 is shown on the distal end of the detachable light housing 500. FIG. 5B shows a front view of the detachable light housing 500 illustrated in FIG. 5A, where the tapered grooves 510 are present on the exterior surface 502 of the housing. The slot 508 is present from the exterior surface 502 to the interior surface 503 of the housing 500 to permit insertion of the tool (not shown). The interior surface 503 comprises ridges or “fingers” 505 which are used to apply pressure to the exterior surface of the tool (not shown). the light sources 504 are arranged around the outer edge of the proximal face 501 of housing 500 in a manner which reduces shadowing due to the presence of elements of the tool (not shown). FIG. 5C shows a side view of the detachable light housing 500 which is illustrated in FIG. 5A. The slot 508 which is present for the length of the housing 500 and which passes all the way through from the exterior surface of the housing to the interior surface is illustrated, including the clamping/holding fingers 505. FIG. 5D shows a break-away side view of the detachable light housing 500, clearly showing a conical shape along the interior surface 503 of the housing 500, with a larger opening 503A being present at the proximal, backside 502 of the housing 500 and a smaller opening 503B being present at the distal, front side (not shown) of the housing 500. The power connection inlet 507 is shown at the proximal, backside of the housing 500. Fingers 505 which provide the clamping action to hold a tool (not shown) are also illustrated.

FIGS. 6A through 6C illustrate the lighting housing 500 from FIG. 5 attached to a variety of surgical or diagnostic device surfaces. FIG. 6A shows the housing 500 attached to a planar surface 610 which may extend from a surgical or diagnostic device (entire device not shown). The internal fingers 505 of the housing hold the planar surface 610 which is inserted through slot 508. FIG. 6B shows a view of the housing 500 attached over a tool 620 having a handle 614, “on”-“off” switches 616, and a front extension 618. FIG. 6A shows the housing 500 attached to a planar surface 610 which may extend from a surgical or diagnostic device (entire device not shown). The internal fingers 505 of the housing hold the planar surface 610 which is inserted through slot 508.

FIG. 6B shows a view of the housing 500 attached over a tool 620 having a handle 614, “on”-“off” switches 616, and a front extension 618. The tool 610 is powered by a power cord 506 which may be attached to a wall outlet (not shown).

FIG. 6C shows the housing 500 attached to an aspirator 630 of the kind used for removing unwanted fluid or smoke from a surgical field. An exterior surface 632 of the aspirator 630 is grasped by fingers 505 on the interior surface of the housing 500. The light sources 504 are designed to have a centerline (not shown) which passes through opening 634 through the interior of the aspirator 630.

FIG. 7 illustrates the considerations which are used in determining the light source spacings which should be used to reduce shadowing for a particular surgical or diagnostic device/tool. The divergence angle is represented by “φ”. “d” represents the diameter of a circle around which the lighting sources are placed. “D” represents a resulting illuminated diameter or spot size at a distance “L” from the light sources. If the divergence angle φ is too great in relation to the distance L, the resulting spot size will be too large, causing a reduction in the illumination intensity or density.

FIG. 7 illustrates the considerations which are used in determining the light source spacings which should be used to reduce shadowing for a particular surgical or diagnostic device/tool. The divergence angle is represented by “φ”. “d” represents the diameter of a circle around which the lighting sources are placed. “D” represents a resulting illuminated diameter or spot size at a distance “L” from the light sources. If the divergence angle φ is too great in relation to the distance L, the resulting spot size will be too large, causing a reduction in the illumination intensity or density. Additionally, as the illumination intensity decreases, the effects of shadowing become significant. Conversely, if the angle φ is too small in relation to the distance L, the resulting spot size will be too small and will not illuminate a large enough area to be useful to the care giver. One of skill in the art, in view of the description provided herein, can design the detachable light to work with an individual surgical or diagnostic device.

In some instances, where there are a series of devices which differ in size, a single detachable light may be capable of working with a number of the devices. For example and not by way of limitation, when the surface to which the light is to be attached is a flat surface, the thickness of the device might range from about 0.02 inch to about 0.12 inch. When the surface to which the light is to be attached is a round surface, the effective diameter of the round surface may range from about 0.03 inch up to about 0.6 inch. For larger sized devices, similar ranges in thickness and effective diameter may be accommodated. These size ranges are provided to enable one of skill in the art to envision how a detachable light can be designed to work with a number of different sized devices, and are not intended to limit the size of the devices with which a detachable light may be employed.

The detachably-mounted compact surgical or diagnostic light structure may be attached and conform automatically to a variety of different and irregular shaped surfaces. This is achieved by integrating three different geometries into the housing.

The first or primary geometry provides high friction axially conical, gripping surfaces with self adjusting lateral clearance. An example of this kind of geometry is illustrated in FIG. 5D, for example, and not by way of limitation. This geometry is well suited to cylindrical, semi-cylindrical, square, and hexagon-shaped instruments and general tools, for example, such as electrocautery pencils, pool suckers, smoke evacuators, and the like.

The second geometry consists of two angled faces. This secondary geometry has the ability to provide a secure grip on thinner planer and semi-planer surfaces such as those found on various types of retractors, for example, but not by way of limitation. Examples of this kind of geometry are shown in FIGS. 2B and 6A, by way of example and not by way of limitation.

The third geometry consists of a conforming arrangement of fingers which are integrated into the housing. This gripping surface provides stable attachment to various instruments such as pickups and retractors, for example, but not by way of limitation.

The attachment geometries described above facilitate easy and immediate removal, relocating or re-attachment of the detachable light from the surgical or diagnostic device.

Many of the internal tissues and structures within the body are sensitive and can be damaged by exposure to the surface of a surgical tool or diagnostic tool. The detachable light needs to provide an exterior surface which is not likely to harm tissue with which it comes into contact. At the same time, contrary to the need for soft atraumatic materials, the housing of the detachable light must maintain dimensional stability of the lighting device, of the lighting elements, of power sources, connectors and such. Further, the functional necessity of generation of a spring force for clamping or attaching the lighting housing onto the surgical instrument of diagnostic device may cause problems.

The conflicting needs described above have been resolved in part by the use of a rigid internal structure fully encased in a soft atraumatic material which makes up the housing's outer surface. For example, the interior of the lighting housing may comprise a ridged (finger-like) internal structure to grip the surgical or diagnostic tool surface and simultaneously provide rigidity of the overall lighting housing structure. The internal ridged members provide structural integrity, alignment for of the lighting housing on the surgical implement or diagnostic tool with which the lighting is designed to work, and the spring force required to create a clamping function to hold the detachable light in place on the implement or tool.

Examples of materials which may be used to form the internal structural features of the housing include nitinol, stainless steels, beryllium copper, and structural plastics such as polyetherimide (PEI), polyoxymethylene, polysulfones poly vinylidene fluoride, nylons, ABS, LCP, and polycarbonate, by way of example and not by way of limitation. Examples of materials which may be used to provide the exterior surface on the housing include silicone, thermoplastic elastomers such as block copolymers of styrene with butadiene or isoprene, and blends of these materials, and latex, by way of example and not by way of limitation.

Managed heat dissipation is important with respect to the detachable light. Typically high intensity light creates a large amount of heat that must be managed and dissipated. Even LED bulbs, which are typically more efficient than other types of light sources when used to create high intensity light, generate a significant amount of heat which must be dissipated. In most embodiments of the invention, this has been resolved without the need for additional heat sinks (which require space), the need for highly thermally conductive materials (which are particularly expensive), or the need for auxiliary cooling (which also requires space).

One method of managing heat dissipation is by the number of light sources which are used, so that each light source is in a high efficiency operating zone. By not driving the individual light sources into their less efficient operating range, less heat is generated. In addition, the shape of the housing for the light sources (bulbs), can be used to reduce heat generation. It is generally known that circular shapes provide the largest amount of surface area to volume ratio. By controlling the diameter of the housing for the light sources (within the maximum sizing necessary for the detachable light tool or diagnostic tool), improved dissipation for unwanted heat can be achieved.

A focused field of view is required to ensure a desired level of uniform luminescence in the target field of view. The present embodiments of the invention achieve this by creating a singular foundation element within the body of the detachable lighting device. The foundation element allows the lighting elements to share a common reference point. This arrangement helps establish and maintain correct orientation and alignment for each element. This ensures the proper direction (X, Y, Z, and angle) for the light output.

To achieve a fully uniform illuminated field, there are a number of elements which must be satisfied. To have a fully illuminated field of view that is properly sized for optimum viewing, the light sources must have the right combination of source location referenced to the field of view, beam divergence a common output direction for the source light, and correct beam overlap. As described above, the location and beam direction can be achieved with the aid of the correct foundation element or elements. Beam divergence can be specified and implemented with lenses used on the light source(s). The remaining element, overlapping pattern of the beams, must be achieved in such a way as to provide sufficient uniform intensity for the resulting spot size.

Minimization of shadowing is very important. In addition to ensuring a fully illuminated field of view, embodiments of the present invention minimize the effects of shadowing caused by the corresponding instrument to which the detachable light is applied. A shadow will always be cast when a light source encounters an obstruction. This effect obviously has a negative impact on a care giver's ability to discern details within the surgical field of view. In order to minimize this effect, the points of light output are arranged in such a way as to create a source mirror on the instrument causing the obstruction. Continuing with the previous eight LED example, LEDs are placed as opposing pairs. There may be some shadows which are created by the presence of elements of the surgical or diagnostic tool cannot be overcome by light placement. While these shadows cannot be removed, they can be offset. By using multiple lights, each zone has light and shadow content but it can be evened out so that the general illumination is uniform. Embodiments of the present invention reduce the effects shadows cause, allowing the care giver to have a highly illuminated field of view.

Embodiments of the present invention where the mounting surface described above is used, provide a self-aligning feature for the light beam direction. This occurs as a result of the co-axial nature of the mounting, tapered geometry of the mounting surface, and carefully arranged symmetry of the light output created by the housing design. This design allows instant alignment simply by placing the device onto an instrument of the kind described above.

If desired, it is possible for the user to make adjustments to the beam direction of any or all of the light sources. This is achieved using a balance between the correct degree of pliability and dimensional material memory for the proximal portion of the housing. Embodiments of the invention employing semi-ridge mounting allow sufficient rigidity to maintain the initial alignment of the light output but not so much rigidity as to prevent movement of the light outputs when a sufficient deliberate force is applied. Once the adjustment force is removed, the semi-ridge mounting again is sufficiently rigid to hold the points of light output in the newly adjusted location until such time as another such deliberate force is applied.

While the foregoing is directed to embodiments of the present invention, other and further embodiments of the invention may be devised in view of the present disclosure, without departing from the basic scope of the invention, and the scope thereof is determined by the claims which follow.

1. A detachable light for use in combination with a tool in surgical or diagnostic applications, said detachable light comprising:
a housing which is attached to an exterior surface of said surgical or diagnostic device;
a plurality of light sources present on an exterior surface of said housing, wherein said plurality of light sources produces a combined luminescent intensity ranging between about 50,000 Lux and about 100,000 Lux, where light source location is managed for heat dissipation; and
a plurality of gripping structures, which grip said exterior surface of said surgical or diagnostic device, said gripping structures located on an interior surface of said housing, wherein at least a portion of said gripping structures provide for alignment of said detachable light housing relative to a centerline of said surgical or diagnostic device.

2. (canceled)

3. A detachable light in accordance with claim 1, wherein an exterior surface of said detachable light housing is formed from an atraumatic material.

4. A detachable light in accordance with claim 3, wherein said atraumatic material is selected from the group consisting of silicone, thermoplastic elastomers and blends thereof, latex, and combinations thereof.

5. A detachable light in accordance with claim 4, wherein said thermoplastic elastomers comprise block copolymers of styrene with butadiene, isoprene, or combinations thereof.

6. A detachable light in accordance with claim 1, wherein said gripping structures comprise a high friction, axially conical primary geometry with a self-adjusting lateral clearance.

7. A detachable light in accordance with claim 6, wherein said gripping structures further comprise a second geometry including two angled faces integrated into said housing.

8. A detachable light in accordance with claim 7, further comprising a third geometry which includes a conforming female rectangular structure integrated into said housing.

9. A detachable light in accordance with claim 1, wherein said plurality of light sources are LED light sources.

10. A detachable light in accordance with claim 9, wherein an intensity of said LED light sources can be adjusted by the user of the detachable light.

11. A detachable light in accordance with claim 9, wherein a frequency of said LED light source ranges from about 3,500° K to about 5,500° K.

12. A detachable light in accordance with claim 9, wherein said plurality of LED light sources comprise more than one frequency of light source.

13. A detachable light in accordance with claim 12, wherein one of said plurality of LED light sources comprises a light source which provides an ultra violet frequency.

14. (canceled)

15. (canceled)

16. A detachable light in accordance with claim 1, wherein said plurality of light sources includes a sufficient number of light sources to enable efficient operation of said light sources, so that the amount of heat generated by said light sources is reduced.

17. A detachable light source in accordance with claim 1 or claim 9, wherein said plurality of light sources includes a sufficient number of light sources to enable efficient operation of said light sources, so that the amount of heat generated by said light sources is reduced.

18. A detachable light in accordance with claim 1, wherein said light source placement facilitates the reduction of shadowing caused by the configurational shape of said surgical or diagnostic device, by creating a light source mirror on an opposite side of said configurational shape which is causing shadowing.

19. A detachable light in accordance with claim 1, wherein adjustments can be made to a beam direction of a light source on the proximal portion of said housing by the application of force by hand to a distal portion of said housing.

20. A detachable light in accordance with claim 1, wherein a power supply which drives said light sources is selected from the group consisting of a battery which is incorporated into said housing, a battery pack attached by cable to said housing, a cable which attaches to a utility supply from a wall receptacle, or a combination thereof.

21. A detachable light in accordance with claim 1, wherein said gripping structures include rigid members.

22. A detachable light in accordance with claim 21, wherein said rigid members are formed from a material selected from the group consisting of nitinol, stainless steel, beryllium copper, polyetherimide, polyoxymethylene, polycarbonate, and combinations thereof.

23. A detachable light for use in combination with a tool in surgical or diagnostic applications, said detachable light comprising:
a housing which is attached to an exterior surface of said surgical or diagnostic device;
a plurality of light sources present on an exterior surface of said housing, wherein said plurality of light sources produces a combined luminescent intensity ranging between about 50,000 Lux and about 100,000 Lux, where light source location is managed for heat dissipation;
a plurality of gripping structures, which grip said exterior surface of said surgical or diagnostic device, said gripping structures located on an interior surface of said housing; and
a semi ridge condition on an exterior surface of said housing which enables a user to adjust or move the location of at least one of said plurality of light sources, so that individual light source elements can be adjusted within a multi-light configuration.

24. A detachable light in accordance with claim 23, wherein said semi ridge condition is present in the form of bendable or malleable light source leads which are connected to a more rigid core which shares or provides the referenced orientation points for said light sources.

25. A detachable light in accordance with claim 23 or claim 24, wherein said at least one light source is movable by application of sufficient force by an operator at particular locations relative to said light source.
